(19) United States
(12) Patent Application Publication
(10) Pub. No.: US 2003/0097075 A1
(43) Pub. Date: May 22, 2003

(54) Automated and remote controlled method and system for assessing function of autonomic nervous system

(76) Inventor: Terry B. J. Kuo, Hualien Hsien (TW)

Correspondence Address: J.C. Patents, Inc., Suite 250, 4 Venture, Irvine, CA, 92618, US

(21) Appl. No.: 09/990,162

(22) Filed: Nov. 20, 2001

(51) Int. Cl. A61B 5/00; A61B 5/02

(52) U.S. Cl. 600/500; 600/301

(57) ABSTRACT

An automated and remote controlled method for assessing the heart rate variability (HRV) is described. Signals that reveal the heart rates of a user are collected and digitized at the Client of a Client/Server system. The digitized signals are then sent to the Server through a network system, where the digitized signals are analyzed on-line with the automated ANS function analysis system to provide indices of the autonomic nervous system. The indices are sent back to the user (the Client) and/or a medical profession for further verification and assistant.

### BACKGROUNDING OF THE INVENTION

1. Field of Invention

The present invention relates to a method and a system for assessing the function of the autonomic nervous systems. More particularly, the present invention relates to an automated and remote controlled method and system for measuring the heart rate variability.

2. Description of Related Art

The autonomic nervous system (ANS) comprises two divisions—the sympathetic and the parasympathetic divisions. Most organs receive impulses from both divisions and under normal circumstances, both divisions work together for a proper organ functioning and adaptation to the demands of life. Problems, including both chronic and acute diseases, arisen when the autonomic nervous system is out of balance, for example, coronary heart disease, hypertension, and even sudden death. Even for the less serious conditions, such as, palpitation, digestive disturbance, breathing difficulty and insomnia, these conditions are believed to be associated with the imbalance of the autonomic nervous system.

Many techniques have been successfully developed to assess the function of the autonomic nervous system. These techniques include heart rate variation with deep breathing, Valsalva response, sudomotor function, orthostatic blood pressure recordings, cold pressor test and biochemistry test, etc. These techniques, however, are mostly invasive and employ expensive diagnostic instruments. These techniques are, therefore, not appropriate for general applications.

The sophistication of the recent computer hardware and software advancements has lead to the development of various techniques for diagnosing the function of the autonomic nervous system. Heart rate variability (HRV), which is a measurement of the beat-to-beat alterations in the heart rate, has been developed as a function indicator for the autonomic nervous system. HRV is an important breakthrough because this technique is non-invasive. Any undue suffering to a patient or a participant of a HRV study is thus prevented. In addition, the hardware for this technique is inexpensive, and thus be broadly applied. Moreover, animal and clinical studies confirm HRV accurately reflects the sympathetic and parasympathetic activities and their balance.

In adult at rest there is about 70 heart beats per minute. These rhythmic heart beats are originated from an electrical event coupling between cardiac muscle cells. The heart receives impulses from both the sympathetic and the parasympathetic divisions of the autonomic nervous system, which normally work together for a homeostatic control of a body. However, if a body is stressed, the sympathetic nervous system dominates causing an increase in heart rate and blood pressure. When the emergency situation has passed the parasympathetic system takes over and decreases the heart rate.

Even under resting conditions, the heart rate of a healthy individual exhibits periodic variations. These periodic variations can be fast, slow, regular or random. The amplitudes of these variations are also rather small, as a result, they are difficult to detect with the conventional analytical methods. However, with the recent advanced development in analytical tools from the field of electrical engineering, a better assessment of HRV by frequency domain analysis, which bases on mathematical manipulations performed on the ECG-derived data, is provided.

Investigators have discovered that, based on frequency analysis, HRV can be characterized into two main components: the high frequency (HF) component and the low frequency (LF) component. The high frequency component is known to be synchronous with respiration and occurs every 3 seconds in a healthy individual. The exact origin of the low frequency component is still under investigation. It is probably related to vessel activity or baroreflex, and occurs about every 10 seconds. Some investigators further divide the low frequency component into a low frequency component and a very low frequency component. Currently, researchers agree that the high frequency (HF) or total power (TP) represents the parasymapthetic control of the heart rate, whereas the ratio LF/HF is considered to mirror the sympathovagal balance or to reflect the sympathetic modulations (Akselrod et al. 1981; Malliani et al 1991). Besides being indicative of the function of the autonomic nervous system, HRV has been documented to reflect other pathological conditions.

Reduced HRV appears to be a marker of an increase of intra-cranial pressure (Lowensohn et al. 1977). Another study further indicates that if the HRV of an elderly is lowered by one standard deviation, his motality rate is 1.7 times higher than a normal individual (Tsjui et al. 1994). Previous studies by the Applicant have demonstrated that LF is eliminated in brain death (Kuo et al. 1997). The effects of aging and gender differences have also been demonstrated to influence the sympathetic and parasympathetic control of the heart rate (Kuo et al. 1999). In the field of gynecology, pregnant women have been reported to have an increased sympathetic function. A more dominant sympathetic and a less effective parasympathetic regulations of the hear rate have been demonstrated to enhance in preeclamptic pregnancy (Yang et al. 2000)

Although the autonomic nervous system (ANS) function provides meaningful reflection of many physiological conditions, the acquisition of information on the ANS function by measuring the heart rate variability (HRV) is not conveniently available to users with the conventional HRV technique. Currently, in order to obtain information on the ANS function, users need to be at a facility where an electrocardiogram (ECG) module is available. The acquisition of ECG signals further requires a proper placing of a plurality of electrodes on various parts of the body. The electrcardio signals are also required to be processed and analyzed by a trained technician to provide meaningful ANS indices. Since such a technique is not user-friendly and is not readily accessible to patients, the application of the conventional technique can not be broadly applied.

### SUMMARY OF THE INVENTION

Accordingly, the present invention provides a method and a system for acquiring indices the autonomic nervous system by measuring heart rate variability (HRV), wherein measurement of signals of the heart rate is readily accessible at a low cost and at the convenient of the user.

Moreover, meaningful results are promptly provided to the user and/or to a medical profession for further verification and assistant, potential adverse consequences are curtailed and the survivability of the user is enhanced.

Accordingly, the present invention provides a method to assess the function of the autonomic nervous system, wherein physiological signals that reveal the cardiac cycle is collected at a first end. The digitized physiological signals are sent to a second end through a network system, where the digitized physiological signals are automatically analyzed to provide the ANS indices. The method further comprises providing the analyzed results to the user and/or a medical profession through the network system. Additionally, the physiological signals that reveal the cardiac cycle collected at the first end according to the present invention comprise the ECG signals, the blood pressure signals, the blood flow signals, the regional blood volume signals, the regional oxygen saturation signals and the heart sound signals using existing physiological function monitoring system, low budget personal computer, personal digital assistant, mobile phone or a microchip.

The present invention provides an automated and remote controlled system for assessing the function of the autonomic nervous system. The automated and remote controlled system comprises a heart rate signal acquisition system to collect the heart rate signals and a diagnosis system to analyze the heart rate signals and to provide the indices of the autonomic nervous system function. The automated and remote controlled system further comprises a network system to transmit the heart rate signals to the diagnosis system from the heart rate signal acquisition system and to transmit the indices of the autonomic nervous system to the heart rate signal acquisition system from the diagnosis system.

Accordingly, the acquisition of the physiological signals that reveal the cardiac cycle is collected at a first end, while the analysis of the physiological signal to obtain meaningful indices is automatically performed at a second end. The collection of physiological signals is thus easily and inexpensively accomplished by users. In other words, the HRV technique is more readily available to patients or even normal subjects. As a result, the data on ANS function can be mass-produced to facilitate the advancement of research on the functional relationship between HRV and the various pathological conditions. Moreover, users can be promptly brought to the attention of his/her health condition to obviate adverse consequences.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a flow diagram illustrating the method in diagnosing the function of the autonomic nervous system according to a preferred embodiment of the present invention;

FIGS. 2A to 2C are illustrative displays of the web site for an automated, remote controlled, ANS function diagnosis system according to the present invention.

FIG. 3 is an example of the frequency spectrum of the various parameters that characterize the heart rate variability.

FIG. 4 shows a comparison between the conventional method in diagnosing the ANS function and the method of the present invention, using electrocardiogram signals as the physiological signals that are collected to determine the heart rate variability.

### DESCRIPTION OF THE PREFERRED EMBODIMENTS

Currently, the majority of the commercialized physiological function monitoring devices already provides digitized signals, for example, devices for monitoring blood pressure, blood flow, regional blood volume, regional oxygen saturation and heart sound. These various types of signals carry information on cardiac cycle, which in turns can be used to determine the heart rate variability. Due to the advancement of the Internet technology, Internet can be used for information transferred in a Client-Server system. Accordingly any instrument that can provide information on the heart rates can be viewed as the Client, whereas the Server provides the service of an instantaneous conversion of the raw physiological signals (including ECG, blood pressure, blood flow, regional blood volume, regional oxygen saturation and heart sound) to meaningful autonomic nervous system (ANS) indices by employing the previously developed automated HRV analysis technique (Kuo et al. 1999). As a result, data on the ANS function can be mass-produced to facilitate the advancement of research on the functional relationship between HRV and the various pathological conditions. Moreover, the HRV technique can be broadly used and readily available to patients or even normal subjects everywhere.

The heart rate variability is typically derived from an ECG (electrocardiogram), which is an electrical recording of the contraction of the cardiac muscles. Besides ECG, blood pressure signals, blood flow signals, regional blood volume signals, regional oxygen saturation signals, heart sound signals and the secondary signals derived from the above signals also contain information on cardiac cycle. These physiological signals, although are not conventionally used to determine HRV, these signals can use as a source of information on HRV to enhance a broader application of the HRV technique.

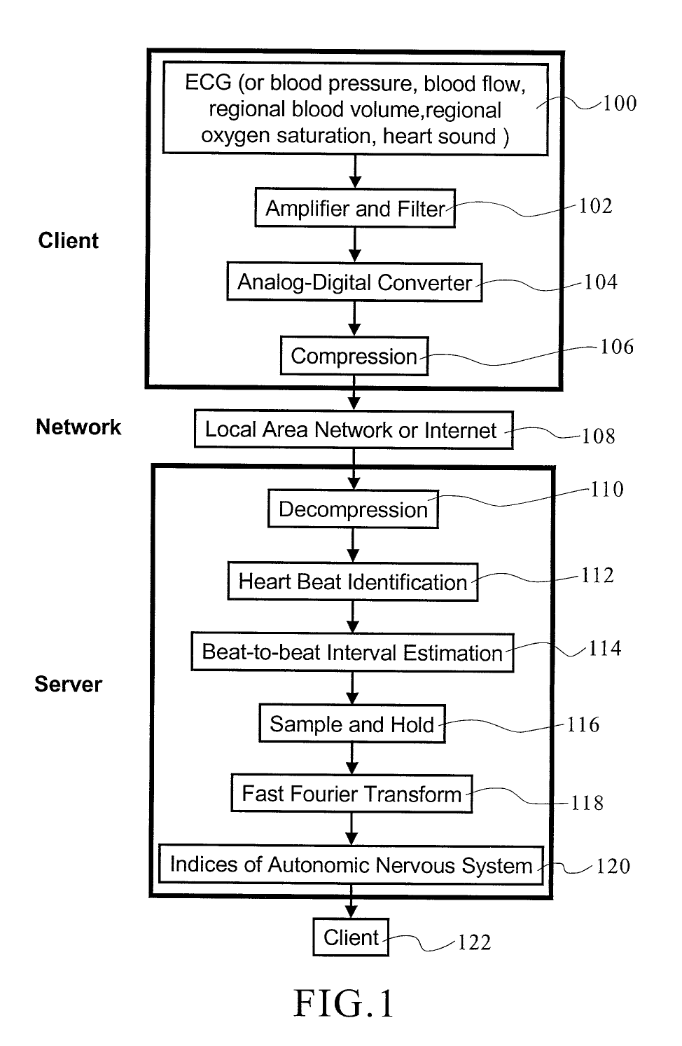

FIG. 1 is a flow diagram illustrating the method in diagnosing the function of the autonomic nervous system according to the present invention.

As shown in FIG. 1, the electrocardiogram, or signals such as, blood pressure, blood flow, regional blood volume, regional oxygen saturation and heart sound flow are collected, for example, for 5 mintues, by an electrode, a transducer or a microphone (step 100). The 5-minute physiological signals are then amplified and filtered with a band pass filter (step 102). The processed signals are further transmitted to an analog-to-digital (A/D) converter with a sampling rate of 256 to 2048 Hz (step 104). The data sampling is accomplished with a computing device, which comprises at least a microprocessor with a sufficient memory. After this, the digitized signals are compressed (step 106) and are sent to the Server through the network for an on-line analysis.

The information transmitted from the Client to the Server is through either local area network or Internet (step 108) via cables, fiber optics or electromagnetic wave.

Still referring to FIG. 1, after the Server receives the digitized physiological signals, such as, ECG, blood pressure, blood flow, regional blood volume, regional oxygen saturation and heart sound, the digitized signals are first decompressed (step 110) and then analyzed to estimate the heart-rate variability. A spike detection algorithm (Kuo and Chan 1992) is used to detect all peaks of the digitized physiological signals. The peak of each heart beat is defined as the time point of the heart beat (step 112), and the interval between two peaks is estimated as the beat-to-beat interval between current and latter heart beats (step 114). Parameters such as amplitude and duration of all peaks are measured so that their means and standard deviations can be calculated as standard templates. Each heart beat is then compared and validated with the standard templates. If the standard score of any of the peak interval values exceeds three, it is considered erroneous and is rejected.

The validated peak interval values are subsequently re-sampled and interpolated at the rate of 7.11 Hz to accomplish the continuity in time domain (step 116). Thereafter, frequency-domain analysis is performed using fast Fourier transform (FFT) (step 118). The DC component of the signals is deleted, and a Hamming window is used to attenuate the leakage effect. For each 288 seconds or 2048 data points, the power spectral density is estimated on the basis of FFT. The resulting power spectrum is corrected for attenuation resulting from the sampling and the Hamming window. The power spectrum is subsequently quantified by means of integration into standard frequency-domain parameters including low-frequency (LF 0.04-0.15 Hz) and high-frequency (HF 0.15-0.40 Hz), total power (TP) and ratio of low frequency to high frequency (LF/HF) (step 120).

The analyzed results, which include the various ANS indices, the frequency spectrums and recommendations, can be sent back to the Client (step 122) through the Network. The user is thereby immediately brought to the attention of his/her health condition. In the case of impairments in the sympathetic and/or parasympathetic functions, whether they are too high or too low, the Server can also design to automatically notify a physician or other medical professions to provide a prompt assistance to the user. According to the present invention, with the rapid diagnosis and transfer of information, and the readily accessibility of HRV measurements to users, potential adverse consequences are curtailed and the survivability of the users is enhanced.

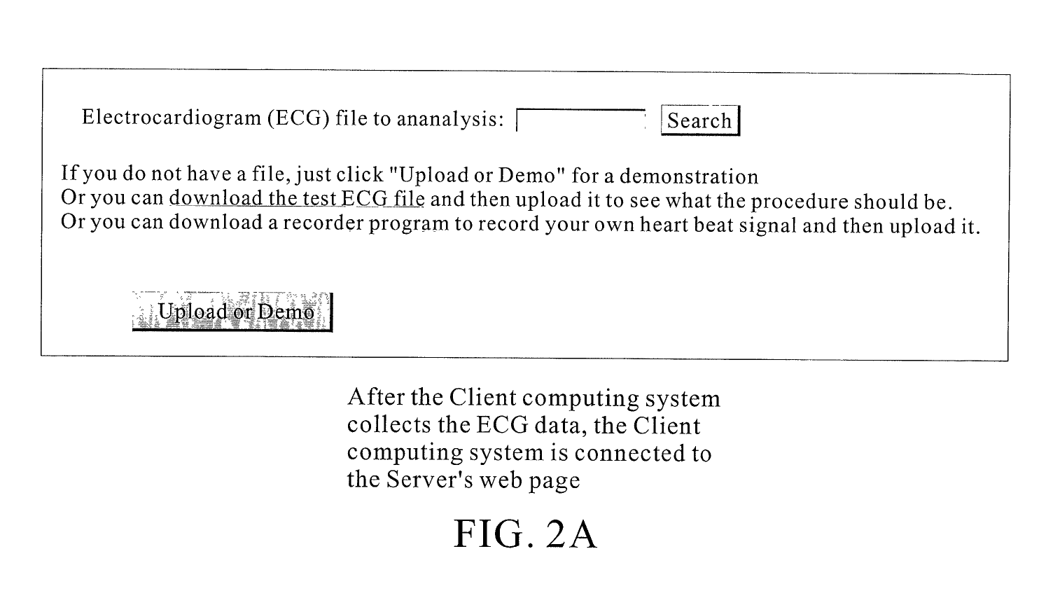

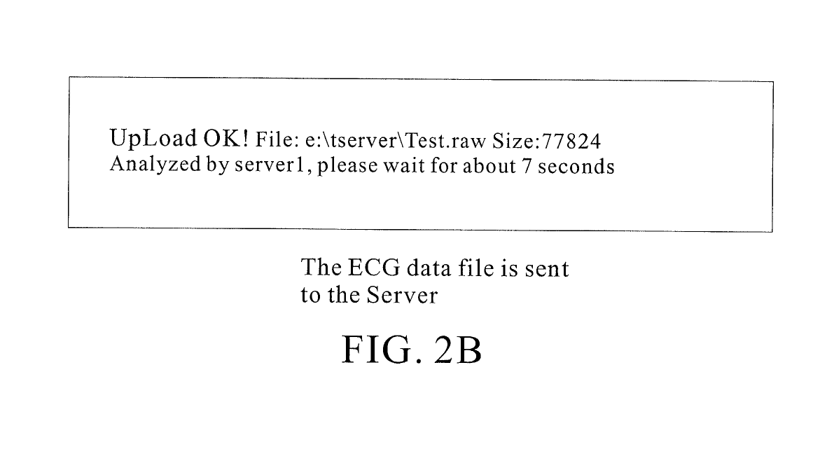

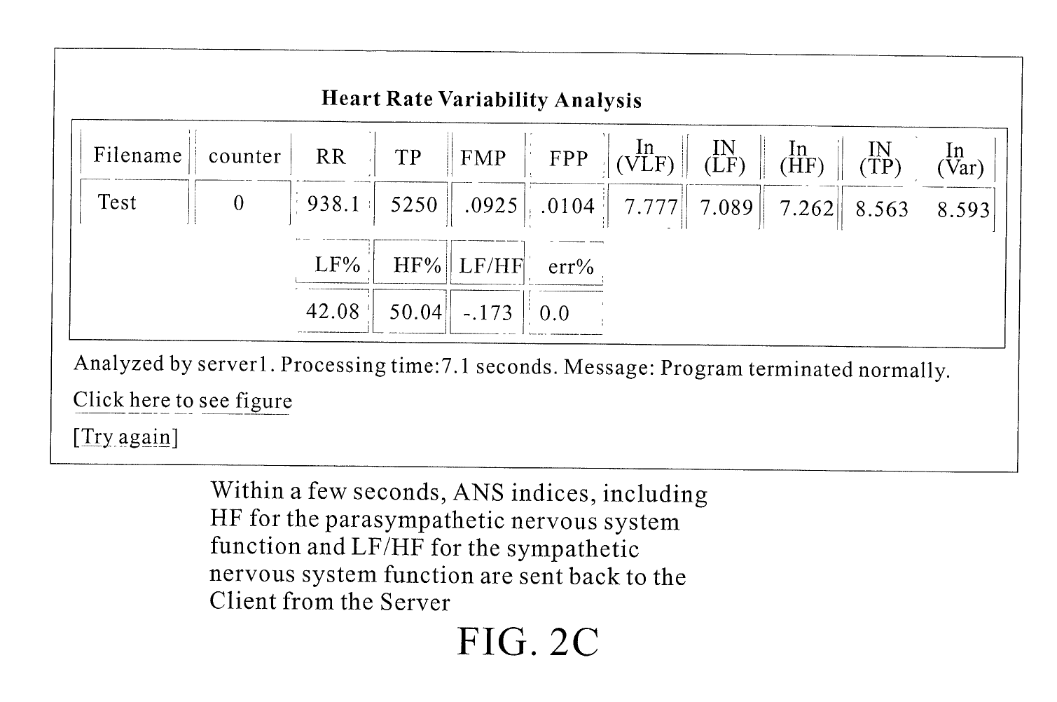

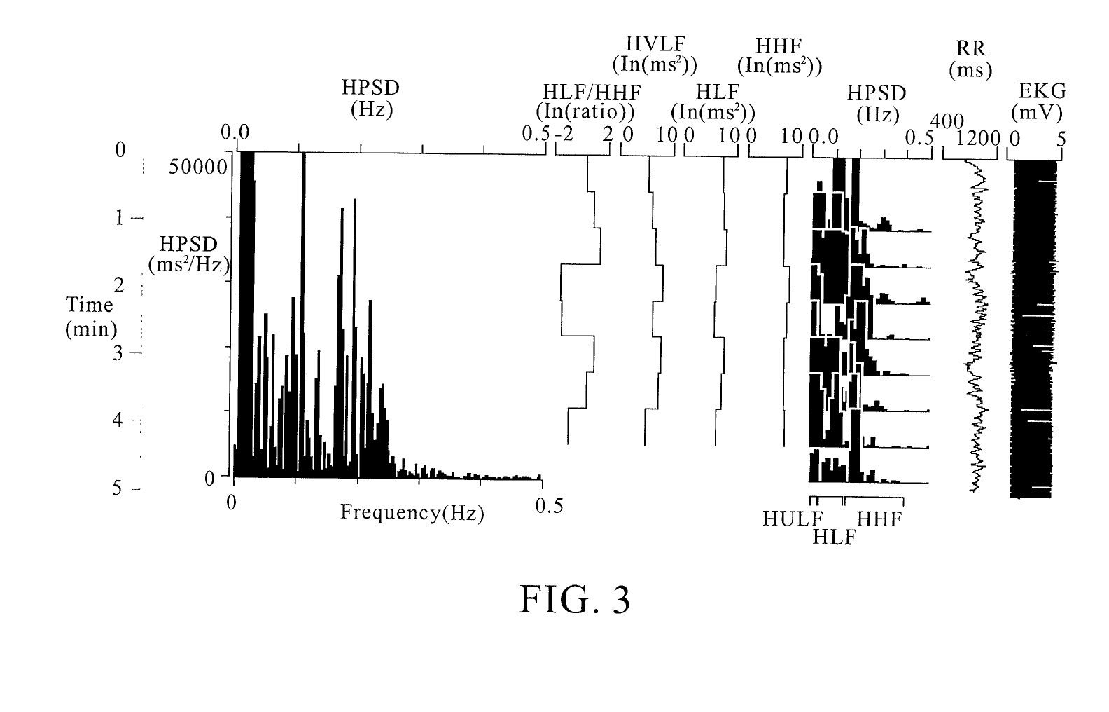

FIGS. 2A to 2C are illustrative displays of the web site for the automated, remote controlled, ANS function diagnosis system according to the present invention. As shown in FIGS. 2A to 2C, the computing system of the Client is connected to the web site of the Server after the collection of the ECG signals. The computer file containing the ECG signals is then sent to the Server. Within a few seconds, the Server replies with the ANS indices, which include HF for the function of the parasympathetic system and LF/HF for the function of the sympathetic system. The Server may further provide the raw frequency spectrums of parameters that characterize the HRV to the Client for an off-line verification.

Many existing physiological function monitoring devices already provide the functions of digitizing the physiological signals, for example, ECG, blood pressure, blood flow, regional blood volume, region oxygen saturation and heart sound, and processing these digitized signals to supply the user with information on the transient or the average heart rate. If these digitized signals are sent to an automated, remote controlled, ANS function diagnosis server through the Internet, the ANS function of a user can be readily available within a few seconds. The digital physiological function monitoring devices for measuring the above heart rate-related signals can thereby be used as the automated, remote controlled ANS function diagnosis system even with no additional software design is provided so as long these devices comprise the network input/output capability.

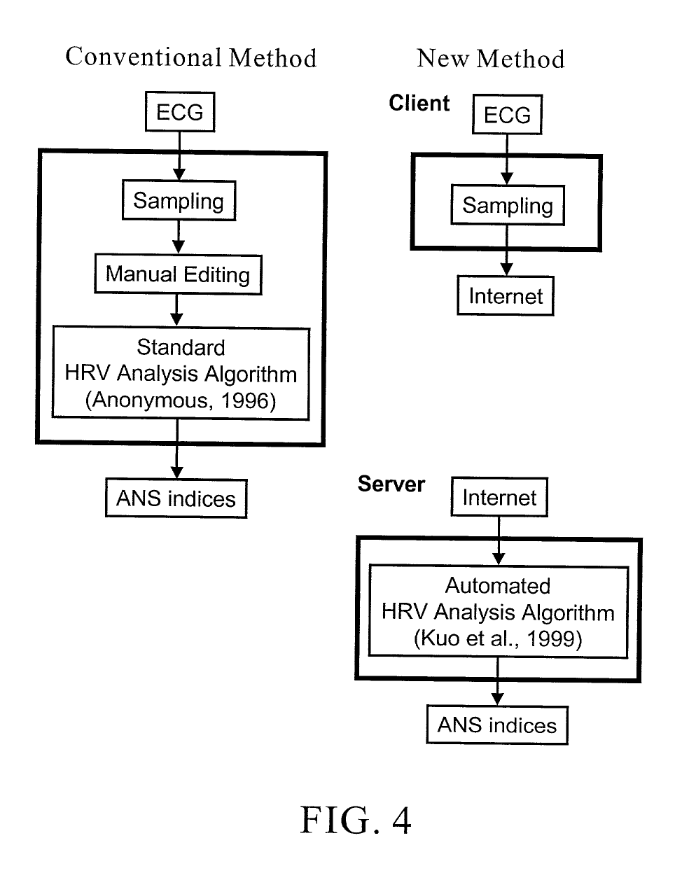

FIG. 4 shows a comparison between the conventional method in diagnosing the ANS function and the method of the present invention, using electrocardiogram signals as the physiological signals. As shown in FIG. 4, the Client is only responsible for data acquisition and output of the data. The analysis of data is not required at the Client. Therefore, only low budget hardware, for example, a physiological function monitoring system, a personal computer, a personal digital assistant (PDA), a mobile phone or a microchip, is required by the Client. As a result, data on the ANS function can be mass-produced to expedite the advancement of research on the functional relationship between HRV and the various pathological conditions. Moreover, the method can be broadly used and readily available to patients or even normal subjects everywhere.

Still referring to FIG. 4, the Server is designed to provide a speedy analysis of the data, for example, by using a high speed computer along and the automated heart rate analysis algorithm (Kuo et al., 1999). An accurate and speedy service is provided to the user by sending back the various ANS indices, converted from the raw physiological signals, to the Client.

Accordingly, the existing physiological monitoring devices provide an additional function, which is for diagnosing the function of the autonomic nervous system. As a result, the cost for the automated, remote controlled, autonomic nervous system function diagnosis system of the present invention is also significantly reduced.

Moreover, since the heart rate signals of the present invention are derived from measurements including blood pressure, blood flow, regional blood volume, regional oxygen saturation, heart sound and ECG, the HRV technique can be more broadly applied. As a matter of fact, some of these measurements can be easily performed by a user (the Client) at the user's own home, using only low budget hardware, such as a personal computer, a PDA, a microchip, etc. for data acquisition. Diagnosing the ANS function is thus readily available at very low cost and at the convenience of the user. Moreover, the present invention also provides an automated, HRV function analysis system (the Server) to receive and to analyze the heart rate signals from the user through a network system and to promptly provide the user with meaningful ANS indices. The user can be immediately brought to the attention of his/her health condition. Since the Server can also design to automatically notify a physician or other medical professions to provide assistant to the user in case of impairments in the sympathetic and/or parasympathetic functions are noted, potential adverse consequences are curtailed and the survivability of the user is enhanced.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A method to assess a function of an autonomic nervous system, comprising:
collecting physiological signals that reveal heart rates at a first end;
sending the physiological signals to a second end through a network system; and
analyzing the physiological signals at the second end, wherein frequency-domain parameters of the physiological signals are automatically quantified to characterize a heart rate variability.

2. The method of claim 1, wherein the physiological signals include digitized physiological signals.

3. The method of claim 1, wherein subsequent to analyzing the digitized physiological signals at the second end, the frequency domain parameters of the physiological signals that are quantified to characterize the heart rate variability are sent back to the first end.

4. The method of claim 1, wherein the second end is designed to notify a third end of the frequency domain parameters of the physiological signals that are quantified to characterize the heart rate variability

5. The method of claim 4, wherein the third end includes a medical profession.

6. The method of claim 1, wherein the physiological signals that reveal the heart rates are selected from the group consisting of an electrocardiogram signal, a blood pressure signal, a blood flow signal, a regional blood volume signal, a regional oxygen saturation signal, a heart sound signal or a secondary signal derived from the physiological signals.

7. The method of claim 1, wherein the physiological signals are collected with an electrode,a transducer or a microphone.

8. The method of claim 1, wherein sending the physiological signals is by using a physiological function monitoring system, a personal computer, a personal digital assistant, a mobile phone or a microchip.

9. The method of claim 1, wherein before sending the physiological signals, the physiological signals are amplified and filtered with a band pass filter.

10. The method of claim 1, wherein the frequency domain parameters include low frequency (LF), high frequency (HF), total power (TP) and LF/HF.

11. The method of claim 1, wherein analyzing the digitized physiological signals at the second end includes performing an automated heart rate variability analysis algorithm.

12. The method of claim 11, wherein the automated heart rate variability analysis algorithm comprises:
estimating beat-to-beat interval values based on the digitized physiological signals;
transforming the interval values into a frequency spectrum; and
quantifying components of a frequency distribution of the heart rate variability.

13. The method of claim 1, wherein the first end includes a Client of a Client/Server system.

14. The method of claim 1, wherein the second end includes a Server of a Client/Server system.

15. The method of claim 1, wherein the network system includes an internet or a local area network.

16. An automated and remote controlled system for assessing a function of an autonomic nervous system (ANS), comprising:
a heart rate signal acquisition system to collect heart rate signals;
a diagnosis system to analyze the heart rate signals, wherein frequency-domain parameters of the heart rate signals are quantified to provide indices of the autonomic nervous system function; and
a network system to transmit the heart rate signals to the diagnosis system from the heart rate signal acquisition system for an on-line analysis and to transmit the indices of the ANS function to the heart rate signal acquisition system from the diagnosis system.

17. The system of claim 16, wherein the heart rate signals that are collected by the heart rate signal acquisition system comprises an electrocardiogram signal, a blood pressure signal, a blood flow signal, a regional blood volume signal, a regional oxygen saturation signal and a heart sound signal.

18. The system of claim 16, wherein the heart rate signal acquisition system further comprises an amplifier, a filter and a computing system to digitize the heart rate signals and to transmit the digitized heart rate signals to the diagnosis system through the network system.

19. The system of claim 16, wherein the heart rate signal acquisition system comprises a physiological function monitoring system, a personal computer, a personal digital assistant, a mobile phone or a microchip.

20. The system of claim 16, wherein the diagnosing system comprises an automated ANS function analysis system to automatically estimate beat-to-beat interval values between current and latter heart beats, transform the interval values into a frequency spectrum and quantify components of a frequency distribution of a heart rate variability.
